(19) United States
(12) Patent Application Publication
(10) Pub. No.: US 2026/0301179 A1
(43) Pub. Date: Oct. 1, 2026

(54) METHOD AND SYSTEM FOR PATIENT ONBOARDING AND DENTAL SCANNING

(71) Applicant: GET-GRIN, INC., Airmont, NY (US)

(72) Inventors: Adam Benjamin SCHULHOF, New City, NY (US); Yarden EILAT-BLOCH, Haifa (IL); Pamela Sharon OREN-ARTZI, Austin, TX (US); Carmi RAZ, Gizo (IL)

(73) Assignee: GET-GRIN, INC., Airmont, NY (US)

(21) Appl. No.: 19/708,474

(22) Filed: Jun. 15, 2026

Related U.S. Application Data
(63) Continuation-in-part of application No. 18/424,237, filed on Jan. 26, 2024.
Application No. 18/424,237 is a continuation of application No. PCT/US2022/038737, filed on Jul. 28, 2022.
(60) Provisional application No. 63/227,076, filed on Jul. 29, 2021.

(51) Int. Cl. G06T 7/00; G16H 10/60; G16H 30/40; G16H 50/20; G16H 80/00

(52) U.S. Cl. CPC G06T 7/0012; G16H 10/60; G16H 30/40; G16H 50/20; G16H 80/00; G06T 2207/10052; G06T 2207/30036

(57) ABSTRACT

Disclosed herein are a system, method, and computer program product embodiments for patient onboarding and generating and editing a dental scan of intraoral and/or extraoral imaging. The embodiments disclosed herein may provide a convenient solution and user experience for dental patients to onboard patients and capture one or more intraoral and/or extraoral videos or images using a mobile device such as a smartphone. The embodiments disclosed herein may provide dentists and orthodontists with a detailed analysis of the patient's dental condition based on one or more full arch scans captured remotely by the patient.

### CROSS REFERENCE TO RELATED PATENT APPLICATIONS

This patent application is a continuation-in-part of U.S. application Ser. No. 18/424,237 entitled “METHOD AND SYSTEM FOR PRESENTING DENTAL SCAN,” filed Jan. 26, 2024, which is a continuation of International Application No. PCT/US2022/038737 entitled “METHOD AND SYSTEM FOR PRESENTING DENTAL SCAN,” filed Jul. 28, 2022, which claims the benefit of U.S. Provisional Patent Application No. 63/227,076 entitled “METHOD AND SYSTEM FOR PRESENTING DENTAL SCAN,” filed Jul. 29, 2021. This patent application is also continuation of International Application No. PCT/US2024/054195, entitled “METHOD AND SYSTEM FOR PATIENT ONBOARDING AND DENTAL SCANNING,” filed Nov. 1, 2024, which claims the benefit of U.S. Provisional Patent Application No. 63/610,227 entitled “METHOD AND SYSTEM FOR PATIENT ONBOARDING AND DENTAL SCANNING,” filed Dec. 14, 2023.

### BACKGROUND

Dental professionals and orthodontists may treat and monitor a patient's dental condition based on in-person visits. Treatment and monitoring of a patient's dental condition may require a patient to schedule multiple in-person visits to a dentist or orthodontist. The quality of treatment and the accuracy of monitoring may vary depending on how often and how consistently a patient sees a dentist or orthodontist. In some cases, suboptimal treatment outcomes may result if a patient is unable or unwilling to schedule regular visits to a dentist or orthodontist.

### BRIEF DESCRIPTION OF THE DRAWINGS

The accompanying drawings are incorporated herein and form a part of the specification.

FIG. 1 is a flowchart illustrating a method for generating a dental scan clip, according to some embodiments.

FIG. 2 is a flowchart illustrating a method for generating a dental model clip, according to some embodiments.

FIG. 3 is a flowchart illustrating a method for remote dental monitoring, according to some embodiments.

FIG. 4 shows a block diagram of a system configured to generate a dental scan clip, generate a dental model clip, enable remote dental monitoring, and enable patient onboarding, according to some embodiments.

FIG. 5A shows an example graphical user interface (GUI) screen for entering information associated with a potential patient, according to some embodiments.

FIG. 5B shows an example GUI screen for inviting a potential patient to join a healthcare practitioner's portal account (or registration system), according to some embodiments.

FIG. 6 is a flowchart for a method for obtaining a dental scan clip, according to some embodiments.

FIG. 7 is an example computer system useful for implementing various embodiments.

In the drawings, like reference numbers generally indicate identical or similar elements. Additionally, generally, the left-most digit(s) of a reference number identifies the drawing in which the reference number first appears.

### DETAILED DESCRIPTION

Provided herein are a system, apparatus, device, method and/or computer program product embodiments, and/or combinations and sub-combinations thereof, for onboarding patients and generating and editing a dental scan of intraoral and/or extraoral imaging. As used herein, intraoral imaging may refer to the acquisition of one or more intraoral videos and/or intraoral images, and extraoral imaging may refer to the acquisition of one or more extraoral videos and/or extraoral images. The embodiments disclosed herein may provide a convenient solution and user experience for dental professionals to onboard patients and capture one or more intraoral and/or extraoral videos or images using a mobile device such as a smartphone. The embodiments disclosed herein may provide dentists and orthodontists with a detailed analysis of the patient's dental condition based on one or more full arch scans captured remotely by the patient or in-clinic by a professional.

Embodiments described herein are also directed to remote dental monitoring solutions to allow dental patients to receive high quality dental care, without requiring a dental professional to be physically present with the patient. Some dental professionals and orthodontists may use conventional teledentistry solutions to accommodate patients'needs and schedules. However, such conventional teledentistry solutions may provide inadequate levels of supervision. Further, such conventional teledentistry solutions may be limited by an inaccurate or insufficient monitoring of a patient's dental condition based on one or more photos taken by the patient, if the photos do not adequately capture various intraoral and/or extraoral features.

In one or more embodiments, a first dental scan of an intraoral region of a patient is obtained utilizing a dental adapter coupled to a camera. A dental scan clip is obtained from the first dental scan based on at least one of a clip start point, a clip end point, and a parameter, the dental scan clip comprising a subset of one or more frames or images from the first dental scan.

In one or more embodiments, a second dental scan of an extraoral region of the patient is obtained utilizing the camera.

In one or more embodiments, the second dental scan comprises a plurality of images, and the plurality of images comprises at least one of a left face profile image of the patient, a right face profile image of the patient, a portrait image of the patient while the patient is smiling, or a portrait image of the patient while the patient is in a resting pose.

In one or more embodiments, at least one first optimal frame or image is obtained from the first dental scan, and at least one second optimal frame or image is obtained from the second dental scan.

In one or more embodiments, information associated with the patient is obtained via a records application. The records application may associate the information associated with the patient together with at least one of the first dental scan, the second dental scan, the at least one first optimal frame or image, or the at least one second optimal frame is associated together via the records application via a patient record. An invitation message is caused, via the records application, to be sent to the patient that enables the patient to register with a portal of a healthcare practitioner.

In one or more embodiments, the records application is configured to prompt the healthcare practitioner to obtain the first dental scan after obtaining the information associated with the patient.

In one or more embodiments, at least one of the clip start point or the clip end point comprises at least one of a maxillary tuberosity or a retromolar pad of the patient.

In one or more embodiments, the parameter comprises at least one of maintaining an upper teeth arch horizontal in at least one first frame or image of the first dental scan, maintaining a lower teeth arch horizontal in at least one second frame or image of the first dental scan, maintaining at least one space between teeth vertical in at least one third frame or image of the first dental scan, maintaining a bite of the patient horizontal with respect to a reference line in at least one fourth frame or image of the first dental scan, or maintaining movement speed of an object in the first dental scan constant.

As used herein, the term “dental scan” refers to a video or an image capture of the intraoral perspective imaging of the teeth arch or of a tooth and/or a video or an image capture of extraoral perspective imaging a user's facial features, including, but not limited to, the teeth arch, one or more teeth, the upper lip, the lower lip, the nose, one or more nostrils, one or more ears, one or more eyes, etc. of a patient.

The term “real-time,” as used herein, generally refers to a simultaneous or substantially simultaneous occurrence of a first event or action with respect to an occurrence of a second event or action. A real-time action or event may be performed within a response time of less than one or more of the following: ten seconds, five seconds, one second, a tenth of a second, a hundredth of a second, a millisecond, or less relative to at least another event or action. A real-time action may be performed by one or more computer processors.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “a,” “an,” and “the,” as used herein, generally refer to singular and plural references unless the context clearly dictates otherwise.

Implementation of the method and system of the present embodiments involves performing or completing selected tasks or steps manually, automatically, or a combination thereof. Moreover, according to actual instrumentation and equipment of particular embodiments of methods, apparatus and systems of the present disclosure, several selected steps may be implemented by hardware or by software on any operating system of any firmware or a combination thereof. For example, as hardware, selected steps of the embodiments described herein may be implemented as a chip or a circuit. As software, selected steps of the embodiments described herein may be implemented as a plurality of software instructions being executed by a computer using any suitable operating system. In any case, selected steps of the method and system of the embodiments described herein may be described as being performed by a data processor, such as a computing platform for executing a plurality of instructions.

### Overview

Embodiments described herein include methods and systems for patient onboarding and intraoral and/or extraoral assessment. As used herein, “intraoral assessment” may refer to assessment of an appearance or condition of an intraoral region of a subject (e.g., a region within the subject's mouth), and “extraoral assessment” may refer to assessment of an appearance or condition of an extraoral region of a subject (e.g., a region outside the subject's mouth). The embodiments disclosed herein can transfer one or more intraoral and/or extraoral videos and/or images taken by users using a mobile device such as a smartphone into a dental scan clip. The embodiments disclosed herein may provide dentists and orthodontists with a detailed analysis of the patient's dental condition based on a video or on one or more images of at least one tooth, such as one or more teeth, one or more dental arches or both dental arches of a subject, captured remotely by a user that has been converted by the present disclosure into a dental scan clip. In some embodiments, the user is the subject. In some embodiments the user is a person other than the subject, present at the same location as the subject and assessment of the captured video or image(s) is performed at a remote location.

As used herein the term “dental scope” or “dental adapter” refer to a device that can be attached or coupled to a camera or mobile phone and enables capturing at least one intraoral image or videos of a subject. An example and possible uses are described in U.S. patent application Ser. No. 17/336,997, which is incorporated by reference herein. The embodiments described herein may be implemented using a software application that is configured to enable a dental patient to capture videos and/or images of intraoral and/or extraoral regions. The software application may be used by a user or a subject (e.g., a dental patient) in conjunction with a mobile device to remotely monitor a dental condition of the subject. The dental condition may comprise a development, a growth, a movement, an appearance, a condition, a physical arrangement, a position, and/or an orientation of the subject's teeth or/and the subject's soft tissues in the oral cavity.

The intraoral adapter may be used for dental scanning for capturing a dental scan clip to be used in remote monitoring. As used herein, “remote monitoring” may refer to monitoring a condition of an intraoral and/or extraoral region of a subject, wherein the monitoring is performed at one or more locations remote from the subject. For example, a dentist or a medical specialist may monitor the intraoral and/or extraoral anatomy or intraoral and/or extraoral condition at a first location that is different than a second location at which the subject is located. The first location and the second location may be separated by a distance spanning at least 1 meter, 1 kilometer, 10 kilometers, 100 kilometers, 1000 kilometers, or more.

The remote monitoring may be performed by assessing an intraoral and/or extraoral condition of the subject using one or more dental scan clips generated from a dental scan captured from the subject when the subject is located remotely from the dentist or a dental office. In some cases, the remote monitoring may be performed in real-time such that a dentist is able to assess the dental condition when a subject uses a mobile device to acquire one or more videos or images of one or more intraoral and/or extraoral regions in the patient's mouth. The remote monitoring may be performed using equipment, hardware, and/or software that is not physically located at a dental office.

As used herein, the term “dental scan clip” may refer to a movie, video or animation, representing an edited capture of intraoral imaging that includes the teeth of the user using the capturing device with a selected start point, the appearance of the user's teeth, and a selected end point.

As used herein, the term “dental model clip” may refer to a movie, video or animation, representing edited capture of 3D data or a 3D model that includes the teeth representation of the user using a presentation tool with a selected start point, the appearance of the 3D model of the teeth and a selected end point.

As used herein, the term “clip starting point” may refer to a particular location in the user's mouth or a 3D data representation of the user's mouth that the dental scan clip or the dental model clip starts.

As used herein, the term “clip end point” may refer to a particular location in the user's mouth or a 3D data representation of the user's mouth that the dental scan clip or the dental model clip ends.

As used herein, the term “3D dental data” may refer to a file or computer-readable data that represents the appearance of a user or generic intraoral imaging.

As used herein, the term “3D model” may refer to a file or computer-readable data that represents the appearance of a user or generic intraoral imaging and can be presented as a copy of user or a generic intraoral copy.

### Dental Scan Clip

In one or more embodiments, a method to generate a dental scan clip is provided. The method comprises: providing a clip start point, a clip end point, and a parameter to keep (e.g., a parameter to be maintained for generation of the dental scan clip); providing a dental scan; editing the dental scan to in accordance with the clip start point and the clip end point while keeping (e.g., maintaining) the parameter.

In one or more embodiments, at least one of the clip start point or the clip end point correspond to an identified maxillary tuberosity or a retromolar pad.

In one or more embodiments, at least one of the maxillary tuberosity or the retromolar pad are visible, for example, in the dental scan clip.

In one or more embodiments, the clip start point and the clip end point correspond to locations of two teeth. That is, each of the clip start point and the clip end point correspond to a respective location of a respective tooth.

In one or more embodiments, the parameter comprises at least one of; an upper teeth arch horizontal in a frame of the dental scan, a lower teeth arch horizontal in the frame, one or more spaces between teeth vertical in the frame, an imaginary line of a teeth arches bite horizontal in the frame (e.g., a subject's bite (i.e., when the user's arches touch) along an imaginary horizontal line), or a constant movement speed of an object.

In one or more embodiments, the dental scan provided is one or more movies or images.

In one or more embodiments, the dental scan provided is a video or image acquired with a mobile device.

In one or more embodiments, the editing comprises changing a scale of at least part of the provided dental scan.

In one or more embodiments, the editing comprises rotation of at least part of the provided dental scan.

In one or more embodiments, the editing comprises changing the frames per second display of at least part of the provided dental scan.

In one or more embodiments, the editing comprises aligning the display of at least part of the provided dental scan.

In one or more embodiments, the editing comprises changing the display perspective of at least part of the provided dental scan.

In one or more embodiments, the editing comprises adding transparent or color effects to at least part of the provided dental scan.

In one or more embodiments, the dental scan clip is a movie, video, or animation.

In one or more embodiments, the dental scan clip is less than 2 minute long, 1.5 minutes long, 1 minute long, 0:45 minutes long (i.e., 45 seconds long), or 0:30 minutes long (e.g., 30 seconds long).

In one or more embodiments, the dental scan clip is more than 0:15 minutes long (e.g., 15 seconds long), 0:30 minutes long (e.g., 30 seconds long), 0:45 minutes long (45 seconds long), or 1 minute long.

In one or more embodiments, the method further comprises presenting the dental scan clip simultaneously with a prior generated dental scan clip.

In one or more embodiments, the method further comprises presenting the dental scan clip overlaying or underlaying with a prior generated dental scan clip.

In one or more embodiments, the differences between the dental scan clip and prior generated dental scan clip are marked.

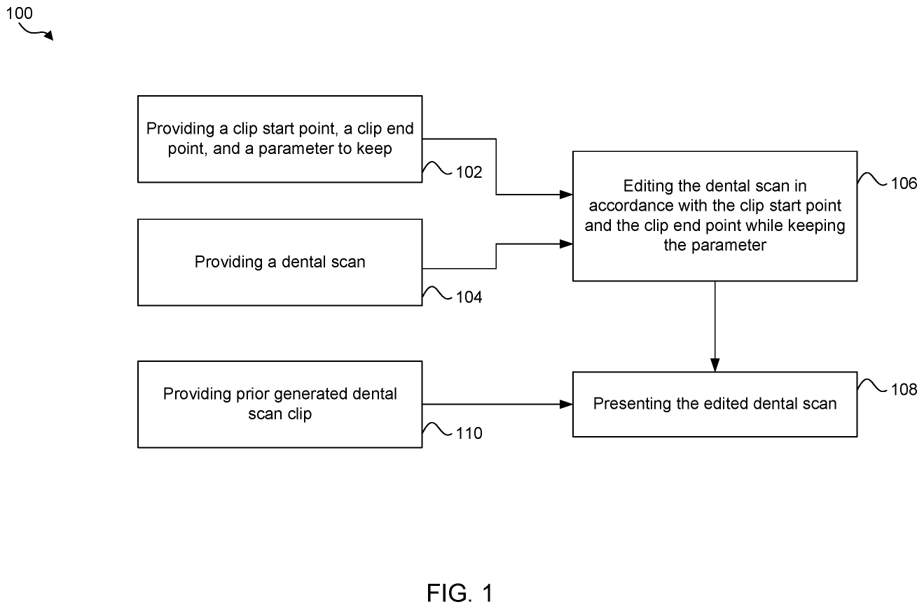

FIG. 1 is a flowchart illustrating a method for generating a dental scan clip, according to some embodiments. Method 100 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 1, as will be understood by a person of ordinary skill in the art. In some aspects, method 100 may be implemented by system 400 (e.g., computing device 404A, computing device 404B, or server(s) 402), as described below. However, method 100 is not limited to that example embodiment.

At 102, a clip start point, a clip end point, and a parameter to keep are provided.

At 104, a dental scan is provided.

At 106, the dental scan may be edited in accordance with the clip start point and the clip end point while keeping the parameter.

At 108, the edited dental scan is presented. In some embodiments, the presenting of the edited dental scan may comprise, at 110, presenting provided prior generated dental scan. That is, a dental scan that was previously-generated (e.g., a dental scan generated at a time previous to generation of the edited dental scan) may be presented in addition to the edited dental scan.

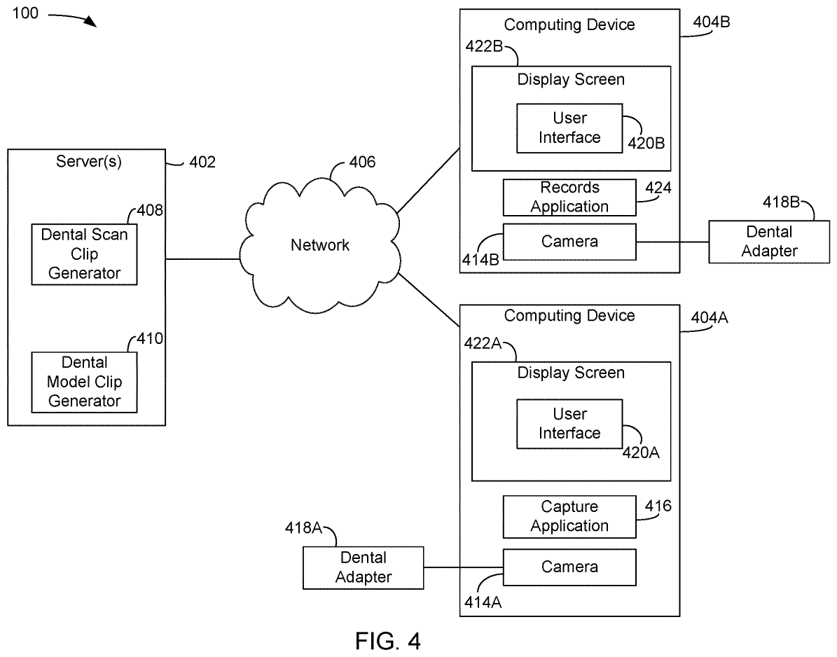

A system to generate a dental scan clip is provided in accordance with the embodiments described herein. An example of such a system is shown below with reference to FIG. 4.

In an embodiment, the dental scan clip may be generated in a mobile device (e.g., computing device 404A or 404B), a remote server (e.g., server(s) 402), or in another computer system. For example, as shown in FIG. 4, the dental scan clip may be generated by dental scan clip generator 408, which may execute on server(s) 402, computing device 404A, computing device 404B, or another computer system.

### Dental Model Clip

In one or more embodiments, a method to transform a three-dimensional (3D) dental data into a dental model clip is provided. The method comprises providing 3D dental data; providing a presentation tool for the 3D dental data; providing perspective acquiring points data; providing a clip start point, a clip end point, and a parameter to keep; and generating a dental model clip in accordance to the clip start point and the clip end point while keeping the parameter.

In one or more embodiments, at least one of the clip start point or the clip end point corresponds to a maxillary tuberosity or a retromolar pad.

In one or more embodiments, the maxillary tuberosity or the retromolar pad are visible, for example, via the dental model clip.

In one or more embodiments, the clip start point and the clip end point are determined to be locations of two teeth. That is, the clip start point may correspond to a location of one tooth, and the clip end point may correspond to a location of another tooth.

In one or more embodiments, the parameter comprises at least one of; an upper teeth arch horizontal in the frame, a lower teeth arch horizontal in the frame, one or more spaces between teeth vertical in the frame, an imaginary line of teeth arches bite horizontal in the frame (e.g., a subject's bite (i.e., when the user's arches touch) along an imaginary horizontal line), or a constant movement speed of an object.

In one or more embodiments, the 3D presentation tool provided can present the 3D dental data as 3D model and enables a user to move and orient the 3D model in different directions along one or more axes (e.g., the x-axis, the y-axis, and/or the z-axis).

In one or more embodiments, the perspective acquiring points data provided are used during the presentation and movement of the 3D model.

In one or more embodiments, the generating comprises changing a scale of at least part of the provided 3D dental data.

In some embodiments the generating comprises a rotation of at least part of the provided 3D dental data.

In one or more embodiments, the generating comprises changing the display perspective of at least part of the provided 3D dental data.

In one or more embodiments, the generating comprises adding transparent or color effects based on the provided 3D dental data.

In one or more embodiments, the dental model clip is a movie, video, or animation.

In one or more embodiments, the dental model clip is less than 2 minute long, 1.5 minute long, 1 minute long, 0:45 minute long (i.e., 45 seconds long), or 0:30 minutes long (e.g., 30 seconds long).

In one or more embodiments, the dental scan clip is more than 0:15 minutes long (e.g., 15 seconds long), 0:30 minutes long (e.g., 30 seconds long), 0:45 minutes long (45 seconds long), or 1 minute long.

In one or more embodiments, the method further comprises presenting the dental model clip simultaneously with a dental scan clip.

In one or more embodiments, the method further comprises presenting the dental model clip overlaying or underlaying with a dental scan clip.

In one or more embodiments, the differences between the dental model clip and dental scan clip are marked.

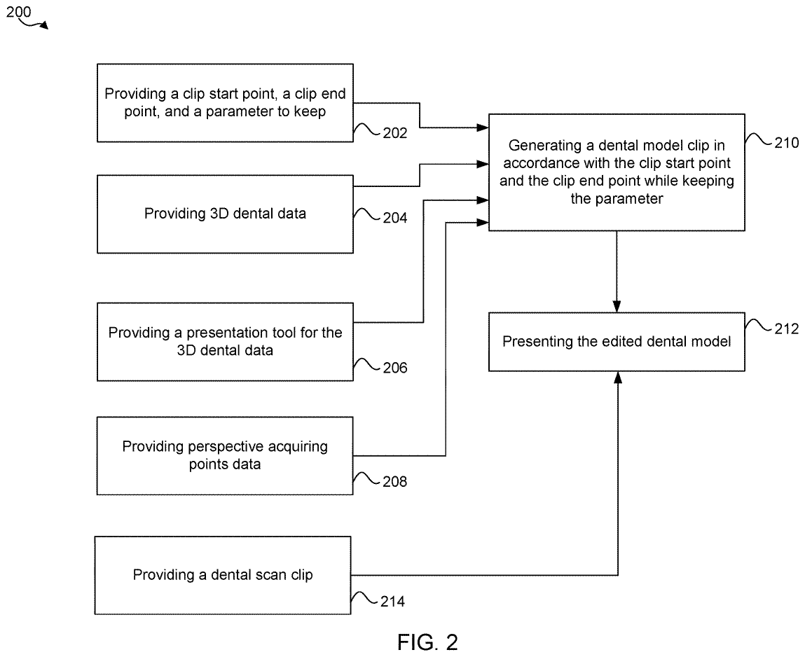

FIG. 2 is a flowchart illustrating method for generating a dental model clip, according to some embodiments. Method 200 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 2, as will be understood by a person of ordinary skill in the art. In some aspects, method 200 may be implemented by system 400 (e.g., computing device 404A, computing device 404B, or server(s) 402), as described below. However, method 200 is not limited to that example embodiment.

At 202, a clip start point, a clip end point, and a parameter to keep may be provided.

At 204, 3D dental data may be provided.

At 206, a presentation tool for the 3D dental data may be provided.

At 208, perspective acquiring points data is provided.

At 210, a dental model clip in accordance with the clip start point and the clip end point while keeping the parameter is generated.

At 212, an edited dental model may be presented. In an embodiment, presenting the edited dental model may comprise providing a dental scan clip (at 214).

A system to generate a dental model clip is provided in accordance with the embodiments described herein. An example of such a system is shown below with reference to FIG. 4.

In an embodiment, the dental model clip may be generated in the mobile device, a remote server or in another computer system. For example, as shown in FIG. 4, the dental model clip may be generated by dental model clip generator 410, which may execute on server(s) 402, computing device 404A, computing device 404B, or another computer system.

### Remote Dental Monitoring

In one or more embodiments, a computer-implemented method for remote dental monitoring is provided. The method comprises: providing a dental scan clip of a user; providing at least one of a prior dental scan clip of the user or a dental model clip of the user; providing a presentation device; and presenting the clips simultaneously.

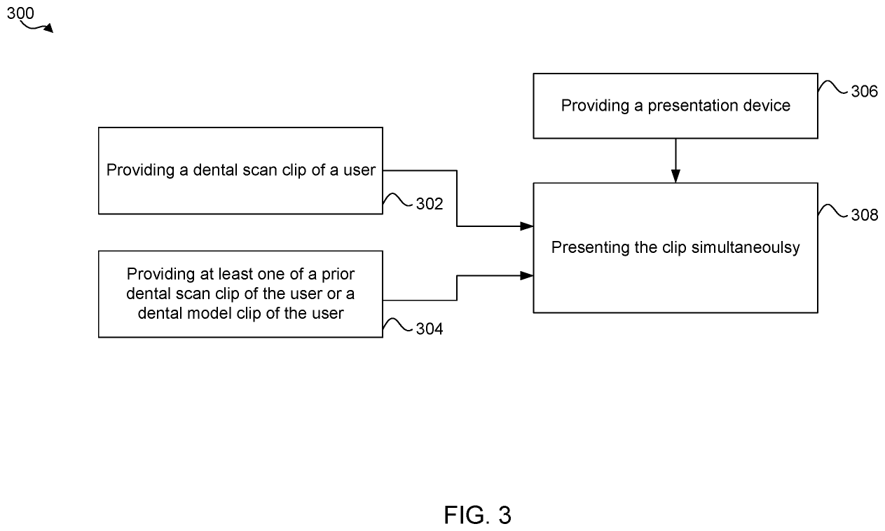

FIG. 3 is a flowchart illustrating method for remote dental monitoring, according to some embodiments. Method 300 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 3, as will be understood by a person of ordinary skill in the art. In some aspects, method 300 may be implemented by system 400 (e.g., computing device 404A, computing device 404B, or server(s) 402), as described below. However, method 300 is not limited to that example embodiment.

At 302, a dental scan clip of a user may be provided.

At 304, at least one of a prior dental scan clip of the user or a dental model clip of the user may be provided.

At 306, a presentation device may be provided.

At 308, the clips may be provided simultaneously.

A system for remote dental monitoring is provided in accordance with the embodiments described herein. An example of such a system is shown below with reference to FIG. 4.

In an embodiment, computing device 404B may utilized to remotely monitor a patient. For example, an application executing on computing device 404B (e.g., records application 424) may obtain a dental scan clip, a prior dental scan clip, and/or a dental model clip from server(s) 402. Such clip(s) may be presented via user interface 420B.

### Example System for Dental Scan Clip Generation, Dental Model Clip Generation, Remote Dental Monitoring, and Patient Onboarding

FIG. 4 shows a block diagram of a system 400 configured to generate a dental scan clip, generate a dental model clip, enable remote dental monitoring, and enable patient onboarding, according to some embodiments. As shown in FIG. 4, system 400 includes one or more servers 402, a computing device 404A, and a computing device 404B. Server(s) 402 and computing devices 404A and 404B may be communicatively coupled to each other via a network 406. Network 406 may comprise one or more networks such as local area networks (LANs), wide area networks (WANs), enterprise networks, the Internet, etc., and may include one or more of wired and/or wireless portions.

In an embodiment, server(s) 402 may form a network-accessible server set (e.g., a cloud-based environment or platform). Server(s) 402 may be accessible via network 406 (e.g., in a “cloud-based” embodiment) to build, deploy, and manage applications and services. Server(s) 402 may be co-located (e.g., housed in one or more nearby buildings with associated components such as backup power supplies, redundant data communications, environmental controls, etc.) to form a datacenter, or may be arranged in other manners. Accordingly, in an embodiment, server(s) 402 may be a datacenter in a distributed collection of datacenters.

Server(s) 402 may be configured to execute one or more software applications (or “applications”) and/or services. Examples of such applications and/or services include dental scan clip generator 408 and dental model clip generator 410. It is noted that while FIG. 4 depicts dental scan clip generator 408 and dental model clip generator 410 as distinct components, dental scan clip generator 408 and dental model clip generator 410 may be integrated into a single component. It is further noted that dental scan clip generator 408 and/or dental model clip generator 410 may be implemented on and executed by computing devices 404A and/or 404B in lieu of server(s) 402. Additional details regarding dental scan clip generator 408 and dental model clip generator 410 are provided below.

Computing device 404A may be utilized by a user, such as a patient or potential patient. As shown in FIG. 4, computing device 404A may comprise a display screen 412A and a camera 414A. Computing device 404A may also be configured to execute a capture application 416. Dental adapter 418A may be attached to camera 414A and enables the capturing of intraoral images and/or videos of the user. An example and possible uses of dental adapter 418A are described in U.S. patent application Ser. No. 17/336,997, which is incorporated by reference herein. Dental adapter 418A may be utilized during execution of capture application 416. Capture application 416, in conjunction with dental adapter 418A, may be configured to enable a dental patient to perform a self-scan of the intraoral region of the dental patient. For example, capture application 416, during execution thereof, may activate camera 414A and may instruct instruction the patient (for example, via a user interface 420A of capture application 416 displayed via a display screen 422A of computing device 404A) to place dental adapter 418A inside the user's mouth. Capture application 416 may provide a command to camera 414A to initiate video capture and/or capture one or more images while dental adapter 418A is inside the user's mouth. The user may move dental adapter 418A and camera 414A in different directions to capture different areas of the patient's intraoral region. The captured video and/or images may be referred to as a dental scan. In an embodiment, capture application 416 may instruct camera 414A to capture video and/or images for a predetermined amount of time, for example, 15 seconds, 30 seconds, 1 minute, 2 minutes, etc. The dental scan may be provided to dental scan clip generator 408, for example, via network 406. Capture application 416 may also be configured to determine and/or generate 3D dental data, for example, based on the dental scan utilizing photogrammetry-based techniques known to persons of ordinary skill in the art. The 3D dental data may be provided to dental model clip generator 410, for example, via network 406. Alternatively, the dental scan may be provided to dental model clip generator 410, and dental model clip generator 410 may be configured to generate the 3D dental data based on the dental scan.

After the dental scan is obtained, dental scan clip generator 408 may be configured to edit the dental scan in accordance with a clip start point, a clip end point, and a parameter. For example, dental scan clip generator 408 may utilize image or object recognition-based techniques and detect a clip start point and a clip end point in the dental scan. In an embodiment, the clip start point and/or the clip end point may comprise the maxillary tuberosity or the retromolar pad of the patient or locations of two different teeth of the patient. Dental scan clip generator 408 may clip (or delete) portions of the dental scan that occur before the clip start point and that occur after the clip end point. Examples of image recognition-based techniques include, but are not limited to, convolutional neural network-based techniques, You Only Look Once (YOLO)-based techniques, etc.

Dental scan clip generator 408 may utilize the parameter to perform various normalizations to the dental scan and/or the clipped dental scan. For example, dental scan clip generator 408 may straighten one or more frames of the video and/or images of the dental scan and/or clipped dental scan with respect to the parameter. The parameter may comprise at least one of maintaining the upper teeth arch horizontal in a particular frame or image of the dental scan and/or clipped dental scan, maintaining the lower teeth arch horizontal in a particular frame or image of the dental scan and/or clipped dental scan, maintaining spaces between teeth vertical in a particular frame or image of the dental scan and/or clipped dental scan, maintaining the subject's bite (i.e., when the teeth arches touch) horizontal with respect to a reference (or imaginary) line in a particular frame or image of the dental scan and/or clipped dental scan, maintaining the movement speed of an object in the dental scan constant (e.g., to correct uneven panning speed to appear smoother), etc. It is noted that the embodiments described herein are not limited to the parameters described herein and that other parameters may be utilized. Server(s) 402 may maintain (e.g., store) the resulting dental scan clip.

Dental model clip generator 410 may be configured to generate a dental model clip based on the 3D dental data, a clip start point, a clip end point, and a parameter. For example, dental model clip generator 410 may utilize image or object recognition-based techniques and detect a clip start point and a clip end point. In an embodiment, the clip start point and/or the clip end point may comprise the maxillary tuberosity or the retromolar pad of the patient or locations of two different teeth of the patient. Dental model clip generator 410 may generate the dental model clip such that the resulting dental model clip starts from the clip start point and ends at the clip end point. Examples of image or object recognition-based techniques include, but are not limited to, convolutional neural network-based techniques, You Only Look Once (YOLO)-based techniques, etc.

Dental model clip generator 410 may utilize the parameter to perform various normalizations to the 3D dental data. For example, dental model clip generator 410 may edit the 3D dental data so that the resulting dental model clip is straight or parallel with respect to one or more reference lines in a similar manner as described above with respect to dental scan clip generator 408. Examples of the parameter may comprise at least one of maintaining the upper teeth arch horizontal, maintaining the lower teeth arch horizontal, maintaining spaces between teeth vertical, maintaining the subject's bite (i.e., when the teeth arches touch) horizontal, etc. It is noted that the embodiments described herein are not limited to the parameters described herein and that other parameters may be utilized. Server(s) 402 may maintain (e.g., store) the resulting dental model clip.

A dentist may utilize computing device 404B to access and view the dental scan clip and/or dental model clip. For example, computing device 404B may comprise an application (e.g., records application 424) that is configured to access the dental scan clip and/or dental model clip from server(s) 402. Records application 424 may further include a user interface 420B (which is displayed via a display screen 422B of computing device 404B) that enables the dentist to view the dental scan clip and/or view and interact with dental model clip.

Computing device 404A may be located in a first location, and computing device 404B may be located in a second location that is different than the first location. This enables the dentist to remotely monitor the patient. The remote monitoring may be performed by assessing an intraoral condition of the subject using the dental scan clip and/or the dental model clip from the subject when the subject is located remotely from the dentist or a dental office. In some cases, the remote monitoring may be performed in real-time such that a dentist is able to assess the dental condition when a subject uses computing device 404A to acquire one or more videos or images and/or a 3D model of one or more intraoral regions in the patient's mouth.

Examples of computing devices 402A and 404B include, but are not limited to any type of stationary device, such as a desktop computer or PC (personal computer), or mobile computing device (such as a laptop computer, a notebook computer, a tablet computer, etc.).

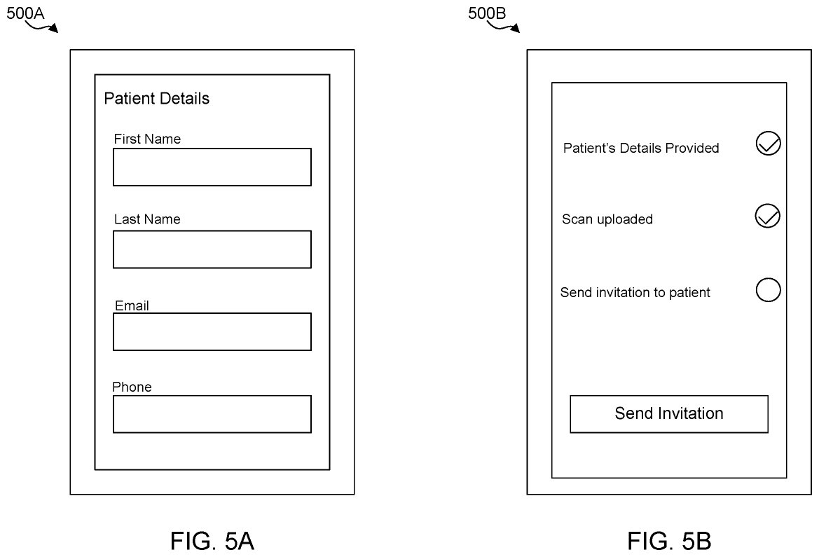

In one or more embodiments, records application 424 may be configured for patient onboarding purposes. The dentist or other practitioner (i.e., a healthcare practitioner) may initiate records application 424, which when initiated, may display a user interface (e.g., user interface 420B) for entering various information associated with the potential patient. FIG. 5A shows an example graphical user interface (GUI) screen 500A for entering information associated with a potential patient, according to some embodiments. As shown in FIG. 5A, GUI screen 500A may comprise a plurality of user interface elements (e.g., text boxes) that enable a user to enter information associated of the potential patient, including, but not limited to, the potential patient's first name, last name, e-mail address, phone number, address, etc. After entering the information, records application 424 may prompt the healthcare practitioner to couple a dental adapter (e.g., dental adapter 418B, which is an example of dental adapter 418A) to camera 414B of computing device 404B and capture a dental scan of the intraoral region of the potential patient. After capturing the dental scan, records application 424 may display a GUI screen that prompts the healthcare practitioner to send an invite to the potential patient to join the healthcare practitioner's portal account. For example, FIG. 5B shows an example GUI screen 500B for inviting a potential patient to join the healthcare practitioner's portal account (or registration system), according to some embodiments. As shown in FIG. 5B, GUI screen 500B may comprise a user interface element (e.g., a button) that, when activated, sends an invitation message to the potential patient's email address.

In one or more embodiments, records application 424 may also prompt the healthcare practitioner to perform extraoral imaging to acquire one or more extraoral videos and/or one or more extraoral images (e.g., a left face profile, a right face profile, a portrait image of the potential patient's face while the potential patient is smiling, a portrait image of the potential patient's resting or passive face, etc.). The healthcare practitioner may utilize camera 414B to obtain the extraoral imaging. The obtained extraoral imaging may be associated with the potential patient's account, along with the intraoral imaging obtained for the potential patient.

In one or more embodiments, dental scan generator 408 may be configured to select the most optimal images from an intraoral dental scan. For example, dental scan generator 408 may be configured to analyze intraoral video and/or images and determine optimal frames from the video and/or optimal images from the images. Optimal frames and/or images may comprise particular features of the intraoral regions. The features may be based on the clip start point, clip end point, and/or parameters described above. For instance, a first optimal frame or image may comprise a left side view of the potential patient's teeth while the arches touch, a second optimal frame or image may comprise a right side view of the potential patient's teeth while the arches touch, a third optimal frame or image may comprise a frontal view of the potential's patient's teeth while the arches touch, a fourth optimal frame or image may comprise a view of the lower arch while the mouth of the potential patient is open, a fifth optimal frame or image may comprise a view of the upper arch while the mouth of the potential patient is open, a sixth optimal frame or image may comprise a frontal view of the potential's patient's mouth while it is open. Dental scan clip generator 408 may utilize various object or image recognition-based techniques known to persons of ordinary skill in the art. Such object or image recognition-based techniques may be configured to detect various intraoral features corresponding to the clip start point, the clip end point, the maxillary tuberosity, and/or the retromolar pad to select particular frames and/or images. Such object or image recognition-based techniques may also be configured to detect other types of intraoral features to select particular frames and/or images. Dental scan clip generator 408 may also be configured to determine the sharpness (or other characteristic) of each of the selected frames and/or images and designate the sharpest frames and/or images as being the most optimal.

In one or more embodiments, dental scan generator 408 may also be configured to select the most optimal images from an extraoral dental scan. For example, dental scan generator 408 may be configured to analyze extraoral video and/or images and determine optimal frames from the video and/or optimal images from the images. Optimal frames and/or images may comprise particular features of the intraoral and/or extraoral regions. The features may be based on the clip start point, clip end point, and/or parameters described above. For instance, a first optimal frame or image may comprise a left face profile (either in a smiling pose or a passive or resting pose), a second optimal frame or image may comprise a right face profile (either in a smiling pose or a passive or resting pose), and a third optimal frame or image may comprise a portrait image of the potential patient's face while the potential patient is smiling, a fourth optimal frame or image may comprise a portrait image of the potential patient's resting or passive face, a fifth optima frame or image may comprise a 45 degree profile. Dental scan clip generator 408 may utilize various object or image recognition-based techniques known to persons of ordinary skill in the art. Such object or image recognition-based techniques may be configured to detect various extraoral and/or intraoral features corresponding to the clip start point, the clip end point, and/or other features (e.g., the upper lip, the lower lip, the nose, one or more nostrils, one or more ears, one or more eyes, etc. of a potential patient) to select particular frames and/or images. Dental scan clip generator 408 may also be configured to determine the sharpness (or other characteristic) of each of the frames and/or images selected from an extraoral scan and designate the sharpest frames and/or images as being the most optimal.

In one or more embodiments, dental scan clip generator 408 may also be configured to generate a composite image based on the most optimal frames and/or images. For example, dental scan clip generator 408 may automatically add certain information to one or more most optimal frames and/or images. Such information includes, but is not limited to, a logo of the dentist's dental practice, the name of the patient, a watermark with the name of the practice, etc. Dental scan clip generator 408 may also be configured to replace one or more images from an intraoral and/or extraoral video for improved records. For example, the image may be a composite image, as described above.

In one or more embodiments, records application 424 may also enable a healthcare practitioner to choose the status of a patient after creating a record thereof. Examples of the patient's status includes, but is not limited, whether the patient is a lead or a paying patient. Records application 424 may also enable a healthcare practitioner to link multiple patient records based on a relationship between different patients. For instance, records application 424 may enable records of different members of a family to be linked together. Records application 424 may also enable a healthcare practitioner to sends documents (e.g., patient records), videos, and/or images to leads before treatment. Records application 424 may also be configured to generate a customized welcome message specific to a particular healthcare practitioner, a patient, or a potential patient. Records application 424 may also enable certain features to be configurable, such as sending invites to potential patients. This way, such features may be skipped during patient onboarding. Records application 424 may also enable a slower scan for record taking purposes. For example, records application 424 may increase the predetermined amount of time that camera 414B is activated, thereby providing more time for the healthcare practitioner to obtain an intraoral dental scan and/or an extraoral dental scan. Records application 424 may also provide automations and tools for better activation rate for patients.

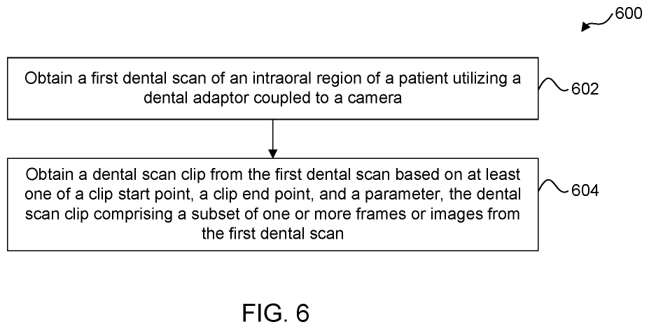

FIG. 6 is a flowchart for a method 600 for obtaining a dental scan clip, according to some embodiments. Method 600 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 6, as will be understood by a person of ordinary skill in the art.

Method 600 shall be described with reference to FIG. 1. However, method 600 is not limited to that example embodiment.

In 602, records application 424 may obtain a first dental scan of an intraoral region of a patient utilizing dental adapter 418B coupled to camera 414B.

In 604, records application 424 may obtain a dental scan clip from the first dental scan based on at least one of a clip start point, a clip end point, and a parameter, the dental scan clip comprising a subset of one or more frames or images from the first dental scan.

In one or more embodiments, records application 424 may obtain a second dental scan of an extraoral region of the patient utilizing camera 414B.

In one or more embodiments, the second dental scan comprises a plurality of images, wherein the plurality of images comprises at least one of a left face profile image of the patient, a right face profile image of the patient, a portrait image of the patient while the patient is smiling, or a portrait image of the patient while the patient is in a resting pose.

In one or more embodiments, records application 424 may obtain at least one first optimal frame or image from the first dental scan, and obtain at least one second optimal frame or image from the second dental scan. For example, records application 424 may provide the dental scan clip to dental scan clip generator 408, which generates the at least one first and second optimal frames or images. Records application 424 may obtain the at least one first and second optimal frames or images from dental scan clip generator 408 and display the at least one first and second optimal frames or images via user interface 420B.

In one or more embodiments, records application 424 may obtain information associated with the patient, associate the information associated with the patient with at least one of the first dental scan, the second dental scan, the at least one first optimal frame or image, or the at least one second optimal frame via a patient record, and cause an invitation message to be sent to the patient that enables the patient to register with a portal of a healthcare practitioner.

In one or more embodiments, records application 424 may be configured to prompt the healthcare practitioner to obtain the first dental scan after obtaining the information associated with the patient.

In one or more embodiments, at least one of the clip start point or the clip end point comprises at least one of a maxillary tuberosity or a retromolar pad of the patient.

In one or more embodiments, the parameter comprises at least one of maintaining an upper teeth arch horizontal in at least one first frame or image of the first dental scan, maintaining a lower teeth arch horizontal in at least one second frame or image of the first dental scan, maintaining at least one space between teeth vertical in at least one third frame or image of the first dental scan, maintaining a bite of the patient horizontal with respect to a reference line in at least one fourth frame or image of the first dental scan, or maintaining movement speed of an object in the dental scan constant.

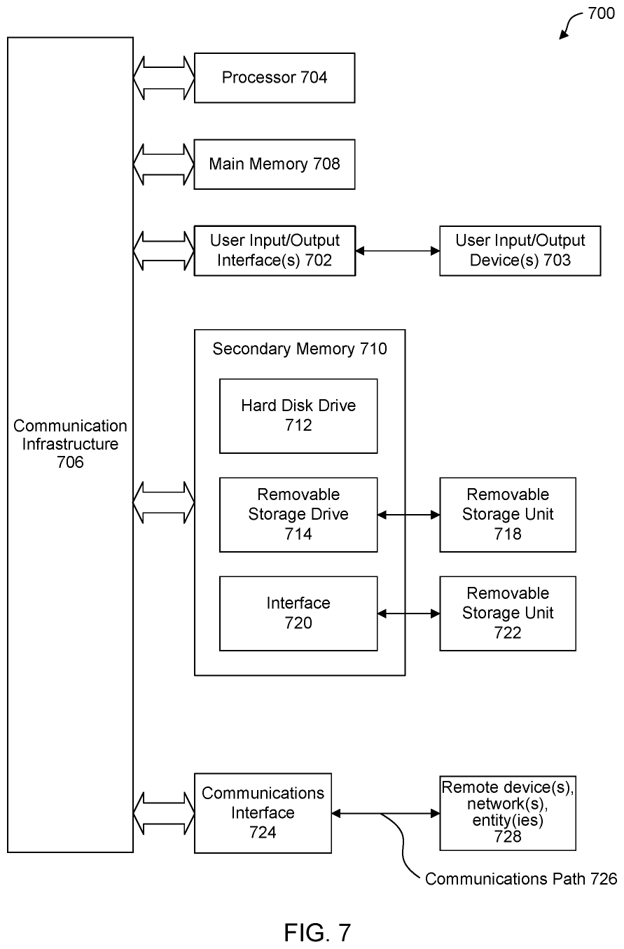

Various embodiments may be implemented, for example, using one or more well-known computer systems, such as computer system 700 shown in FIG. 7. One or more computer systems 700 may be used, for example, to implement any of the embodiments discussed herein, as well as combinations and sub-combinations thereof.

Computer system 700 may include one or more processors (also called central processing units, or CPUs), such as a processor 704. Processor 704 may be connected to a communication infrastructure or bus 706.

Computer system 700 may also include user input/output device(s) 703, such as monitors, keyboards, pointing devices, etc., which may communicate with communication infrastructure 706 through user input/output interface(s) 702.

One or more of processors 704 may be a graphics processing unit (GPU). In an embodiment, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 700 may also include a main or primary memory 708, such as random access memory (RAM). Main memory 708 may include one or more levels of cache. Main memory 708 may have stored therein control logic (i.e., computer software) and/or data.

Computer system 700 may also include one or more secondary storage devices or memory 710. Secondary memory 710 may include, for example, a hard disk drive 712 and/or a removable storage device or drive 714. Removable storage drive 714 may be a floppy disk drive, a magnetic tape drive, a compact disk drive, an optical storage device, tape backup device, and/or any other storage device/drive.

Removable storage drive 714 may interact with a removable storage unit 718. Removable storage unit 718 may include a computer usable or readable storage device having stored thereon computer software (control logic) and/or data. Removable storage unit 718 may be a floppy disk, magnetic tape, compact disk, DVD, optical storage disk, and/any other computer data storage device. Removable storage drive 714 may read from and/or write to removable storage unit 718.

Secondary memory 710 may include other means, devices, components, instrumentalities or other approaches for allowing computer programs and/or other instructions and/or data to be accessed by computer system 700. Such means, devices, components, instrumentalities or other approaches may include, for example, a removable storage unit 722 and an interface 720. Examples of the removable storage unit 722 and the interface 720 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 700 may further include a communication or network interface 724. Communication interface 724 may enable computer system 700 to communicate and interact with any combination of external devices, external networks, external entities, etc. (individually and collectively referenced by reference number 728). For example, communication interface 724 may allow computer system 700 to communicate with external or remote devices 728 over communications path 726, which may be wired and/or wireless (or a combination thereof), and which may include any combination of LANs, WANs, the Internet, etc. Control logic and/or data may be transmitted to and from computer system 700 via communication path 726.

Computer system 700 may also be any of a personal digital assistant (PDA), desktop workstation, laptop or notebook computer, netbook, tablet, smart phone, smart watch or other wearable, appliance, part of the Internet-of-Things, and/or embedded system, to name a few non-limiting examples, or any combination thereof.

Computer system 700 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 700 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some embodiments, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 700, main memory 708, secondary memory 710, and removable storage units 718 and 722, as well as tangible articles of manufacture embodying any combination of the foregoing. Such control logic, when executed by one or more data processing devices (such as computer system 700), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 7. In particular, embodiments can operate with software, hardware, and/or operating system implementations other than those described herein.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

What is claimed is:

1. A computer-implemented method, comprising:
obtaining, by at least one computer processor, a first dental scan of an intraoral region of a patient utilizing a dental adapter coupled to a camera; and
obtaining a dental scan clip from the first dental scan based on at least one of a clip start point, a clip end point, and a parameter, the dental scan clip comprising a subset of one or more frames or images from the first dental scan.

2. The computer-implemented method of claim 1, further comprising obtaining a second dental scan of an extraoral region of the patient utilizing the camera.

3. The computer-implemented method of claim 2, wherein the second dental scan comprises a plurality of images, wherein the plurality of images comprises at least one of:
a left face profile image of the patient;
a right face profile image of the patient;
a portrait image of the patient while the patient is smiling; or
a portrait image of the patient while the patient is in a resting pose.

4. The computer-implemented method of claim 2, further comprising:
obtaining at least one first optimal frame or image from the first dental scan; and
obtaining at least one second optimal frame or image from the second dental scan.

5. The computer-implemented method of claim 4, further comprising:
obtaining, via a records application, information associated with the patient;
associating, via the records application, the information associated with the patient with at least one of the first dental scan, the second dental scan, the at least one first optimal frame or image, or the at least one second optimal frame via a patient record; and
causing, via the records application, an invitation message to be sent to the patient that enables the patient to register with a portal of a healthcare practitioner.

6. The computer-implemented of claim 5, wherein the records application is configured to prompt the healthcare practitioner to obtain the first dental scan after obtaining the information associated with the patient.

7. The computer-implemented method of claim 1, wherein at least one of the clip start point or the clip end point comprises at least one of a maxillary tuberosity or a retromolar pad of the patient.

8. The computer-implemented method of claim 1, wherein the parameter comprises at least one of:
maintaining an upper teeth arch horizontal in at least one first frame or image of the first dental scan;
maintaining a lower teeth arch horizontal in at least one second frame or image of the first dental scan;
maintaining at least one space between teeth vertical in at least one third frame or image of the first dental scan;
maintaining a bite of the patient horizontal with respect to a reference line in at least one fourth frame or image of the first dental scan; or
maintaining movement speed of an object in the first dental scan constant.

9. A system, comprising:
one or more memories; and
at least one processor each coupled to at least one of the one or more memories and configured to perform operations comprising:
obtaining a first dental scan of an intraoral region of a patient utilizing a dental adapter coupled to a camera; and
obtaining a dental scan clip from the first dental scan based on at least one of a clip start point, a clip end point, and a parameter, the dental scan clip comprising a subset of one or more frames or images from the first dental scan.

10. The system of claim 9, wherein the operations further comprise obtaining a second dental scan of an extraoral region of the patient utilizing the camera.

11. The system of claim 10, wherein the second dental scan comprises a plurality of images, and wherein the plurality of images comprises at least one of:
a left face profile image of the patient;
a right face profile image of the patient;
a portrait image of the patient while the patient is smiling; or
a portrait image of the patient while the patient is in a resting pose.

12. The system of claim 10, the operations further comprising:
obtaining at least one first optimal frame or image from the first dental scan; and
obtaining at least one second optimal frame or image from the second dental scan.

13. The system of claim 12, the operations further comprising:
obtaining, via a records application, information associated with the patient;
associating, via the records application, the information associated with the patient with at least one of the first dental scan, the second dental scan, the at least one first optimal frame or image, or the at least one second optimal frame via a patient record; and
causing, via the records application, an invitation message to be sent to the patient that enables the patient to register with a portal of a healthcare practitioner.

14. The system of claim 13, wherein the records application is configured to prompt the healthcare practitioner to obtain the first dental scan after obtaining the information associated with the patient.

15. The system of claim 9, wherein at least one of the clip start point or the clip end point comprises at least one of a maxillary tuberosity or a retromolar pad of the patient.

16. The system of claim 9, wherein the parameter comprises at least one of:
maintaining an upper teeth arch horizontal in at least one first frame or image of the first dental scan;
maintaining a lower teeth arch horizontal in at least one second frame or image of the first dental scan;
maintaining at least one space between teeth vertical in at least one third frame or image of the first dental scan;
maintaining a bite of the patient horizontal with respect to a reference line in at least one fourth frame or image of the first dental scan; or
maintaining movement speed of an object in the first dental scan constant.

17. A non-transitory computer-readable medium having instructions stored thereon that, when executed by at least one computing device, cause the at least one computing device to perform operations comprising:
obtaining a first dental scan of an intraoral region of a patient utilizing a dental adapter coupled to a camera; and
obtaining a dental scan clip from the first dental scan based on at least one of a clip start point, a clip end point, and a parameter, the dental scan clip comprising a subset of one or more frames or images from the first dental scan.

18. The non-transitory computer-readable medium of claim 17, wherein the operations further comprise obtaining a second dental scan of an extraoral region of the patient utilizing the camera.

19. The non-transitory computer-readable medium of claim 18, wherein the second dental scan comprises a plurality of images, and wherein the plurality of images comprises at least one of:
a left face profile image of the patient;
a right face profile image of the patient;
a portrait image of the patient while the patient is smiling; or
a portrait image of the patient while the patient is in a resting pose.

20. The non-transitory computer-readable medium of claim 18, the operations further comprising:
obtaining at least one first optimal frame or image from the first dental scan; and
obtaining at least one second optimal frame or image from the second dental scan.
